(19) United States
(12) Patent Application Publication
(10) Pub. No.: US 2024/0000866 A1
(43) Pub. Date: Jan. 4, 2024

(54) NOVEL STREPTOCOCCUS THERMOPHILUS STRAIN AND PROBIOTIC COMPOSITION AND USE THEREOF

(71) Applicant: NUTRAREX BIOTECH CO., LTD., Taichung City (TW)

(72) Inventors: Meei-Yn Lin, Taichung City (TW); Pin-Chao Huang, Taichung City (TW); Pei-Cheng Lin, Taichung City (TW); Tsung-Han Lu, Taichung City (TW)

(21) Appl. No.: 18/152,641

(22) Filed: Jan. 10, 2023

(30) Foreign Application Priority Data
Jun. 30, 2022 (TW) 111124627

(51) Int. Cl. A61K 35/744; C12N 1/20; C12P 19/26; A61P 27/04; A61P 29/00; A61P 39/06; A23L 33/135

(52) U.S. Cl. CPC A61K 35/744; C12N 1/205; C12P 19/26; A61P 27/04; A61P 29/00; A61P 39/06; A23L 33/135; C12R 2001/46

(57) ABSTRACT

The present disclosure provides a novel Streptococcus thermophilus strain, and the probiotic composition thereof and the use thereof for producing sialic acid and hyaluronic acid, anti-oxidation, anti-inflammatory, and alleviating dry eye syndrome. The novel Streptococcus thermophilus strain and/or metabolites thereof of the present disclosure can be used to prepare medicaments, food products, health food, and/or external products for anti-oxidation, anti-inflammation, and alleviating dry eye syndrome.

### CROSS-REFERENCE TO RELATED APPLICATION

This application claims priority of Taiwan patent application No. 111124627, filed on Jun. 30, 2022, the content of which is incorporated herein in its entirety by reference.

### STATEMENT REGARDING SEQUENCE LISTING

The sequence listing associated with this application is provided in text format in lieu of a paper copy and is hereby incorporated by reference into the specification. The name of the XML file containing the sequence listing is 111F0683-IE Sequence listing. The XML file is 10000 bytes; was created on Oct. 25, 2022.

### BACKGROUND OF THE INVENTION

### 1. Field of the Invention

The present invention relates to a novel Streptococcus thermophilus strain and the probiotic composition and uses thereof. In particular, the present invention relates to a novel Streptococcus thermophilus strain which produces sialic acid and/or hyaluronic acid, the probiotic composition thereof, and methods for anti-oxidation and anti-inflammation by using the novel Streptococcus thermophilus strain.

### 2. The Prior Art

Bird's nest is traditionally used for anti-aging, skin health, pregnant women conditioning, and immune regulation. The main functional ingredient in bird's nest is sialic acid, which is the general term for the derivatives of amino acids or hydroxyl hydrogens substituted in neuraminic acid. Sialic acid is an important component of mucus, glycoproteins, and gangliosides, so it is very important for the normal development of cell membranes, cell membrane receptors, and the brain, and it is widely present in animal tissues, especially in bird's nest, there is a large amount of sialic acid. Sialic acid has physiological functions such as improving intelligence and memory, anti-senile dementia, anti-virus, improving intestinal absorption of vitamins and minerals, and immune regulation. As the understanding and applicability of sialic acid bioactivity improves, so does the demand for sialic acid. However, obtaining sialic acid from bird's nest is not only time-consuming, but also prone to industrial pollution due to the use of non-polar liquids in the extraction method. Therefore, sialic acid is extracted from food-derived substances such as milk or egg yolk, but the yield and purity of this method are low, and it cannot be effectively applied to the production of sialic acid.

Hyaluronic acid (HA) belongs to a linear glycosaminoglycan, which is widely distributed in connective tissue, epithelial tissue, and nerve tissue of animals, such as skin, eyes, and joints with the highest concentration, and is the main component of the extracellular matrix. Hyaluronic acid has the functions of wound healing, soothing joint discomfort, increasing skin elasticity and moisturizing. The content of hyaluronic acid would gradually decrease with age, so as age increases; additional supplementation is required to maintain better physiological function. In the past, the source of hyaluronic acid was mainly extracted from non-edible Streptococcus zooepidemicus or cockscomb. However, the literature points out that the high protein content of cockscomb extract may lead to allergic reactions, and non-edible Streptococcus must be completely sterilized and purified before it can be used in products, and does not have the function of probiotics.

In summary, sialic acid and hyaluronic acid have rich functional properties, but there is no functional product that is safe and has both at the same time. Therefore, it is indeed necessary to find a new strain that is derived from the human body and can be well adapted to the gastrointestinal tract environment and is safe, can produce sialic acid and hyaluronic acid in large quantities, and is safe and can be colonized in the body at the same time, so as to increase the content of sialic acid and hyaluronic acid in the body to achieve the use of beauty and health care.

### SUMMARY OF THE INVENTION

In order to solve the foregoing problems, an objective of the present invention is to provide a Streptococcus thermophilus iHA318 strain, deposited in Deutsche Sammlung von Mikroorganismen und Zellkulturen (DSMZ) under an accession number DSM 33978, wherein the Streptococcus thermophilus iHA318 strain produces sialic acid.

Another objective of the present invention is to provide a probiotic composition, comprising a Streptococcus thermophilus iHA318 strain and/or its metabolites, wherein the Streptococcus thermophilus iHA318 strain is deposited in Deutsche Sammlung von Mikroorganismen und Zellkulturen (DSMZ) under an accession number DSM 33978.

According to an embodiment of the present invention, the Streptococcus thermophilus iHA318 strain is cultivated using a medium, and the medium comprises a nutrient source which is a small molecular nitrogen source. Preferably, the small molecular nitrogen source is urea. More preferably, the small molecular nitrogen source is 0.4-0.6% urea.

According to an embodiment of the present invention, the Streptococcus thermophilus iHA318 strain is viable, inactive or its metabolites.

Another objective of the present invention is to provide a method for producing sialic acid and/or hyaluronic acid by using the aforementioned probiotic composition.

According to an embodiment of the present invention, the probiotic composition is a medicament, a nutritional supplement, a health food, a food product, a skin health product, an external product, or a combination thereof.

Another objective of the present invention is to provide a method for anti-oxidation and/or anti-inflammation, comprising administering to a subject in need thereof a pharmaceutical composition comprising an effective amount of the aforementioned probiotic composition.

According to an embodiment of the present invention, the probiotic composition enhances antioxidant ability of a nerve cell and/or an immune cell.

According to an embodiment of the present invention, the probiotic composition reduces an inflammatory response of an immune cell.

Another objective of the present invention is to provide a method for preventing and/or treating dry eye syndrome, comprising administering to a subject in need thereof a pharmaceutical composition comprising an effective amount of the aforementioned probiotic composition.

According to an embodiment of the present invention, the effective amount is 1×10⁸-1×10¹¹ CFU of the Streptococcus thermophilus iHA318 strain.

According to an embodiment of the present invention, the pharmaceutical composition is a medicament, a nutritional supplement, a health food, a food product, a skin health product, an external product, or a combination thereof.

According to an embodiment of the present invention, the pharmaceutical composition may further comprise a pharmaceutically acceptable excipient, carrier, adjuvant, and/or food additive. In addition, the dosage form of the pharmaceutical composition can be a solution, a suspension, a semi-solid preparation, a solid preparation, a gelatin capsule, a soft capsule, a lozenge, a pill, a syrup, a lozenge, a tablet, a chewing gum, and/or a freeze-dried powder preparation.

Another objective of the present invention is to provide a method for producing sialic acid and/or hyaluronic acid by using the aforementioned probiotic composition.

The present invention provides a novel Streptococcus thermophilus iHA318 strain, and the optimum culture formulation of the Streptococcus thermophilus iHA318 strain is measured and urea is used as a nutrient nitrogen source. The optimum concentration is 0.4-0.6% urea, which can greatly improve the activity of the iHA318 strain and greatly increase the number of bacterial growth.

The Streptococcus thermophilus iHA318 strain and/or its metabolites of the present invention can effectively improve the cell viability of nerve cells and immune cells in oxidative damage, has excellent antioxidant activity for nerve cells and immune cells, and therefore can be prepared as a pharmaceutical composition for anti-oxidation. The Streptococcus thermophilus iHA318 strain and/or its metabolites of the present invention can also effectively reduce the inflammatory response induced by immune cells, so it has excellent anti-inflammatory activity for immune cells, and can be prepared as an anti-inflammatory pharmaceutical composition.

In addition, compared to the Streptococcus thermophilus ST002 strain also isolated from breast milk and the conventional Streptococcus thermophilus BCRC 14017 strain, the novel Streptococcus thermophilus iHA318 strain of the present invention can more effectively secrete sialic acid and hyaluronic acid, and the hyaluronic acid secreted covers a wide range of molecular weights. Therefore, the novel Streptococcus thermophilus iHA318 strain of the present invention can be applied not only to the production of sialic acid and hyaluronic acid, but also to skin moisturizing, immune regulation, dry eye syndrome, anti-inflammatory and anti-oxidative purposes.

### BRIEF DESCRIPTION OF THE DRAWINGS

The following drawings form part of the present specification and are included here to further demonstrate some aspects of the present invention, which can be better understood by reference to one or more of these drawings, in combination with the detailed description of the embodiments presented herein.

FIG. 1 shows the result of bacterial count analysis of the Streptococcus thermophilus iHA318 strain of the present invention in a medium containing different nutrient sources.

FIG. 2 shows the result of bacterial count analysis of the Streptococcus thermophilus iHA318 strain of the present invention in a medium containing different concentrations of urea.

FIG. 3 shows the efficacy result of the Streptococcus thermophilus iHA318 strain of the present invention on enhancing the antioxidant ability of nerve cells.

FIG. 4 shows the efficacy result of the Streptococcus thermophilus iHA318 strain of the present invention on enhancing the antioxidant ability of immune cells.

FIG. 5 shows the efficacy result of the Streptococcus thermophilus iHA318 strain of the present invention on enhancing the anti-inflammatory ability of immune cells.

FIG. 6 shows the efficacy result of the Streptococcus thermophilus iHA318 strain of the present invention on alleviating dry eye syndrome.

FIG. 7 shows the result of the large amount of sialic acid secreted by the Streptococcus thermophilus iHA318 strain of the present invention compared to the conventional strain.

FIG. 8 shows the result of the large amount of hyaluronic acid secreted by the Streptococcus thermophilus iHA318 strain of the present invention compared to the conventional strain.

FIG. 9 shows the result of molecular weight analysis of hyaluronic acid secreted by the Streptococcus thermophilus iHA318 strain of the present invention.

FIG. 10 shows the experimental procedure of the animal experiment of the Streptococcus thermophilus iHA318 strain of the present invention on alleviating dry eye syndrome, in which TBUT represents tear film break up time; UVB damage: irradiate UVB for a certain seconds per day in units of 0.72 J/cm²; Tear volume: tear test.

FIG. 11 shows that the Streptococcus thermophilus iHA318 strain of the present invention significantly restores tear secretion capacity, in which *p<0.05 compared with Damage group.

FIG. 12 shows that the Streptococcus thermophilus iHA318 strain of the present invention gradually restores tear film function, in which *p<0.05 compared with Damage group.

FIG. 13 shows that the Streptococcus thermophilus iHA318 strain of the present invention repairs and maintains eye transmittance, in which *p<0.05 compared with Damage group.

FIG. 14 shows that the Streptococcus thermophilus iHA318 strain of the present invention repairs pupil surface smoothness and maintains clear vision, in which *p<0.05 compared with Damage group.

FIG. 15 shows that the Streptococcus thermophilus iHA318 strain of the present invention repairs and maintains the smoothness of the entire ocular surface, and protects the health of the ocular surface in an all-round way, in which *p<0.05 compared with Damage group.

FIG. 16 shows that the Streptococcus thermophilus iHA318 strain of the present invention repairs ocular surface damage, in which *p<0.05 compared with Damage group.

### DETAILED DESCRIPTION OF THE PREFERRED EMBODIMENT

In the following detailed description of the embodiments of the present invention, reference is made to the accompanying drawings, which are shown to illustrate the specific embodiments in which the present disclosure may be practiced. These embodiments are provided to enable those skilled in the art to practice the present disclosure. It is understood that other embodiments may be used and that changes can be made to the embodiments without departing from the scope of the present invention. The following description is therefore not to be considered as limiting the scope of the present invention.

As used herein, the data provided represent experimental values that can vary within a range of ±20%, preferably within ±10%, and most preferably within ±5%.

Excel software is used for statistical analysis. Data are presented as mean±standard deviation (SD), and differences between them are analyzed by Student's t-test.

The “Probiotic or Probiotic bacteria” described herein is a microorganism whose cells, mixed strains, extracts, or metabolites have a positive effect on the host. The probiotic bacteria are usually derived from the individual and are beneficial to the individual's health, and can also refer to certain microorganisms that are supplemented by external sources and may be beneficial to the individual.

The “metabolites” described herein are substances secreted by a microorganism after being metabolized. More specifically, they can be substances secreted by bacteria into the bacterial culture medium during culture. In the present invention, the metabolites of the Streptococcus thermophilus iHA318 strain may comprise sialic acid and hyaluronic acid.

The scientific name “Streptococcus thermophilus” described herein is equivalent to “Streptococcus salivarius subsp. Thermophilus”.

According to the present invention, the operating procedures and parameter conditions related to bacterial culture fall within the scope of the professional literacy and routine techniques of those skilled in the art.

According to the present invention, the operating procedures and parameter conditions related to the analysis of antioxidant activity fall within the scope of the professional literacy and routine techniques of those skilled in the art.

According to the present invention, the operating procedures and parameter conditions related to the analysis of anti-inflammatory activity fall within the scope of the professional literacy and routine techniques of those skilled in the art.

According to the present invention, the operating procedures and parameter conditions related to the analysis of purification of sialic acid and its content fall within the scope of the professional literacy and routine techniques of those skilled in the art.

According to the present invention, the operating procedures and parameter conditions related to the analysis of purification of hyaluronic acid and its content and molecular weight fall within the scope of the professional literacy and routine techniques of those skilled in the art.

Unless otherwise stated herein, the term “treatment” should not be construed as treating an individual until complete recovery, but should include maintaining an individual's disease progression or symptoms to a substantially static degree, increasing an individual's recovery rate, the improvement of the severity of a specific condition, or the improvement of an individual's life quality.

The present invention provides a novel Streptococcus thermophilus iHA318 strain, which is a probiotic strain with the ability to secrete sialic acid and hyaluronic acid and has antioxidant and anti-inflammatory effects. The Streptococcus thermophilus iHA318 strain of the present invention is isolated from breast milk samples, and is determined to be Streptococcus thermophilus by API® 50 CHL microbial identification Kit (BioMerieux). The conventional method is also used to extract the total RNA of the Streptococcus thermophilus iHA318 strain of the present invention and reverse transcription is carried out, and 16S rDNA is used as target gene to carry out bacterial identification. Three sets of different universal primers were used to amplify the 16S rDNA of the reverse transcription product of the total RNA by polymerase chain reaction (PCR), and then the successfully amplified PCR products with longer length were sequenced and decoded. The three sets of primers are shown in Table 1. The methods for extracting total RNA and reverse transcription are well known to those with ordinary skill in the art, and would not be repeated here. The nucleic acid fragment obtained by sequencing is shown in SEQ ID NO: 7. Then the nucleic acid fragment was compared with the 16S rRNA gene sequences of other strains of Streptococcus thermophilus in the GenBank of the National Center for Biotechnology Information (NCBI) in the United States, and the sequences are more than 99% identical. Therefore, it is confirmed that the iHA318 strain of the present invention is Streptococcus thermophilus (Streptococcus salivarius subsp. Thermophilus or Streptococcus thermophilus).

TABLE 1

| | Universal | SEQ ID | |
|---|---|---|---|
| Group | primer | NO | Sequences |
| 1 | 27F | SEQ ID | AGAGTTTGATCMTGGCTCAG |
| | | NO: 1 | |
| | 1525R | SEQ ID | AAGGAGGTGWTCCARCC |
| | | NO: 2 | |
| 2 | 8F2 | SEQ ID | TGGAGAGTTTGATCCTGGCTCAG |
| | | NO: 3 | |
| | 806R | SEQ ID | GGACTACCAGGGTATCTAAT |
| | | NO: 4 | |
| 3 | FD1 mod | SEQ ID | AGAGTTTGATCYTGGYTYAG |
| | | NO: 5 | |
| | 16S1RR-B | SEQ ID | CTTTACGCCCARTRAWTCCG |
| | | NO: 6 | |

The physiological characteristics of the Streptococcus thermophilus iHA318 strain of the present invention are as follows. Growth temperature is 35° C. to 40° C. Growth pH<4.6 after 24 hours of growth in appropriate medium. Oxygen effects are facultative anaerobic. The culture method of the Streptococcus thermophilus iHA318 strain of the present invention is as follows. The iHA318 strain was inoculated on agar medium (De Man, Rogosa and Sharpe, MRS) or M17 liquid medium and cultured at 37° C. in an anaerobic environment. The appearance characteristics of the colonies of the Streptococcus thermophilus iHA318 strain of the present invention are as follows. The edge is complete, the colony is slightly white and larger, and the surface is smooth and raised. The morphological characteristics of the Streptococcus thermophilus iHA318 strain of the present invention are as follows: streptococcal shape, no sporulation, and no mobility. The Gram staining result of the Streptococcus thermophilus iHA318 strain of the present invention is positive.

The Streptococcus thermophilus iHA318 strain of the present invention was deposited in the Biosource Collection and Research Center (BCRC) of the Food Industry Research and Development Institute (FIRDI) on Mar. 11, 2022, under accession number BCRC 911114. Meanwhile, the Streptococcus thermophilus iHA318 strain of the present invention was deposited in the Deutsche Sammlung von Mikroorganismen and Zellkulturen (DSMZ) on Aug. 17, 2021, under accession number DSM 33978.

Compared with the conventional Streptococcus thermophilus BCRC 14017 strain, the Streptococcus thermophilus iHA318 strain of the present invention has effects of antioxidant and anti-inflammatory activity. Based on these beneficial biological activities, the Streptococcus thermophilus iHA318 strain of the present invention is expected to have the potential to be used to enhance an individual's antioxidant and/or anti-inflammatory properties. Therefore, the present invention further provides a probiotic composition comprising the Streptococcus thermophilus iHA318 strain of the present invention.

The probiotic composition of the present invention can be applied to the purposes of preparing a pharmaceutical composition. The pharmaceutical composition is used to enhance the anti-inflammatory and/or antioxidant ability of an individual. The pharmaceutical composition can be a medicament, a nutritional supplement, a health food, a food product, a skin health product, an external product, or a combination thereof. The pharmaceutical composition can further comprise a pharmaceutically acceptable excipient, carrier, adjuvant, and/or food additive.

In a preferred embodiment of the present invention, the probiotic composition of the present invention is formulated in a pharmaceutically acceptable vehicle and made into a dosage form suitable for oral administration, and the pharmaceutical composition is preferably in a dosage form selected from the group consisting of: solution, suspension, powder, tablet, pill, syrup, lozenge, troche, chewing gum, capsule, and the like.

According to the present invention, the pharmaceutically acceptable vehicle can comprise one or more reagents selected from the group consisting of solvent, buffer, emulsifier, suspending agent, decomposer, disintegrating agent, dispersing agent, binding agent, excipient, stabilizing agent, chelating agent, diluent, gelling agent, preservative, wetting agent, lubricant, absorption delaying agent, liposome, and the like. The selection and quantity of these reagents fall within the scope of the professional literacy and routine techniques of those skilled in the art.

According to the present invention, the pharmaceutically acceptable vehicle comprises a solvent selected from the group consisting of water, normal saline, phosphate buffered saline (PBS), aqueous solution containing alcohol, and combinations thereof.

According to the present invention, the skin health product may further comprise an acceptable adjuvant that is widely used in skin health product manufacturing techniques. For example, the acceptable adjuvant may comprise one or more reagents selected from the group consisting of: solvent, gelling agent, active agent, preservative, antioxidant, screening agent, chelating agent, surfactant, coloring agent, thickening agent, filler, fragrance, and odor absorber. The selection and quantity of these reagents fall within the scope of the professional literacy and routine techniques of those skilled in the art.

In another preferred embodiment of the present invention, the probiotic composition of the present invention can be prepared as a food product, and is formulated with edible materials to include but not limited to: beverages, fermented foods, bakery products, health foods, nutritional supplements, and dietary supplements.

According to the present invention, the edible material is selected from the group consisting of: water, fluid milk products, milk, concentrated milk; fermented milk such as yogurt, sour milk, frozen yogurt, and lactic acid bacteria-fermented beverages; milk powder; ice cream; cream cheeses; dry cheeses; soybean milk; fermented soybean milk; vegetable-fruit juices; juices; sports drinks; confectionery; jellies; candies; infant formulas; health foods; animal feeds; Chinese herbals; and dietary supplements.

According to the present invention, the food product can be regarded as a food additive, which is added during the preparation of raw materials by conventional methods, or added in the production process of food, and formulated with any edible material into food products for human and non-human animals to eat.

### Cell Culture Method

In the embodiment of the present invention, the test of nerve cells is carried out using PC12 cells. PC12 cells were purchased from the Food Industry Research and Development Institute, with the number BCRC 60048. Culture is performed in RPMI-1640 medium (Sigma) supplemented with 2 mM glutamine (Sigma), 1.5 g/L sodium bicarbonate (Sigma), 4.5 g/L glucose (Sigma), 10 mM 4-(2-hydroxyethyl)-1-piperazineethanesulfonic acid (HEPES; Sigma), 1 mM sodium pyruvate (Sigma), 100 U/mL penicillin/streptomycin (Sigma), 10% horse serum (Hyclone, Logan, UT, USA), and 5% fetal bovine serum (FBS; Gibco). The culture method of PC12 cells is as follows: 1.5×10⁴/well cells are seeded in a 96-well culture dish, each well containing 100 μL of culture medium, and cultured in a 5% CO₂, 37° C. environment.

In the embodiment of the present invention, the test of immune cells is carried out using murine monocyte macrophages (hereinafter referred to as Raw264.7 cells). Raw264.7 cells were purchased from the Food Industry Research and Development Institute, with the number BCRC 60001. Culture was performed using Dulbecco's modified eagle medium (DMEM) (Gibco) supplemented with 10% fetal bovine serum (Gibco), 1% L-glutamic acid (Gibco), and 1% penicillin-streptomycin amphoteric, AA (Gibco). The culture method of Raw264.7 cells is as follows: 1×10⁴/well cells are seeded in a 96-well culture dish, each well containing 100 μL of culture medium, and cultured in a 5% CO₂, 37° C. environment.

The following would describe in detail: the optimum culture formulation of the Streptococcus thermophilus iHA318 strain of the present invention, and efficacy test of the probiotic composition of the present invention for enhancing the antioxidant ability of nerve cells and enhancing the antioxidant and anti-inflammatory abilities of immune cells. The differences in the ability of the Streptococcus thermophilus iHA318 strain of the present invention and the conventional strain to secrete sialic acid and hyaluronic acid are compared, to confirm that the probiotic composition of the present invention has the desired effect, and can be used to prepare a composition with corresponding effect.

### Example 1

Optimum Culture Formulation of Streptococcus thermophilus iHA318 Strain of Present Invention

In one embodiment of the present invention, in order to obtain the formulation most suitable for culturing the Streptococcus thermophilus iHA318 strain of the present invention, the difference caused by the growth medium of different nutrient sources (nitrogen sources) to the growth of the iHA318 strain is firstly examined, to find out the optimum nutrient source of the iHA318 strain. The optimum nutrient source is then configured in the culture medium with different concentrations to examine the difference caused by the concentration of the nutrient source to the growth of the iHA318 strain, to find out the optimum nutrient source concentration of the iHA318 strain.

First, the original bacteria of the Streptococcus thermophilus iHA318 strain of the present invention in the frozen tube were thawed, and 1 mL of bacterial liquid was taken out and 9 mL of sterile water was added. After standing for 1 hour, μL of the diluent was taken and added to 10 mL of media A to G in Table 2. All components of media A to G are the same, except that the 5 g nutrient source in media B to G is skimmed milk powder, tryptic soy, urea, meat extract, yeast extract, and casein. After quantifying with sterile water to a total volume of 1 L, incubation was performed at 37° C. for 24 hours. After mixing the bacterial liquids of different media evenly, 10 mL of the bacterial liquids were taken, 90 mL of sterile water was added for serial dilution to the appropriate concentration. 1 mL of the diluent was taken out and the pour culture method was used to culture on MRS agar based on anaerobic culture under 37° C. for 48 hours, followed by bacterial count analysis.

TABLE 2

| Medium number | A | | B | | C | | D | | E | | F | | G | |
|---|---|---|---|---|---|---|---|---|---|---|---|---|---|---|
| Proteose Peptone | 10 | g | 10 | g | 10 | g | 10 | g | 10 | g | 10 | g | 10 | g |
| No. 3 | | | | | | | | | | | | | | |
| Beef Extract | 10 | g | 10 | g | 10 | g | 10 | g | 10 | g | 10 | g | 10 | g |
| Yeast Extract | 5 | g | 5 | g | 5 | g | 5 | g | 5 | g | 5 | g | 5 | g |
| Magnesium Sulfate | 0.1 | g | 0.1 | g | 0.1 | g | 0.1 | g | 0.1 | g | 0.1 | g | 0.1 | g |
| Dextrose (Glucose) | 20 | g | 20 | g | 20 | g | 20 | g | 20 | g | 20 | g | 20 | g |
| Polysorbate 80 | 1 | g | 1 | g | 1 | g | 1 | g | 1 | g | 1 | g | 1 | g |
| Ammonium Citrate | 2 | g | 2 | g | 2 | g | 2 | g | 2 | g | 2 | g | 2 | g |
| Sodium Acetate | 5 | g | 5 | g | 5 | g | 5 | g | 5 | g | 5 | g | 5 | g |
| Manganese Sulfate | 0.05 | g | 0.05 | g | 0.05 | g | 0.05 | g | 0.05 | g | 0.05 | g | 0.05 | g |
| Dipotassium | 2 | g | 2 | g | 2 | g | 2 | g | 2 | g | 2 | g | 2 | g |

| | | | | | | | |
|---|---|---|---|---|---|---|---|
| Phosphate | | | | | | | |
| 5 g nutrient source | none | skimmed | tryptic soy | urea | meat extract | yeast extract | casein |
| | | milk powder | | | | | |

| | |
|---|---|
| H₂O | Quantify to 1 L |

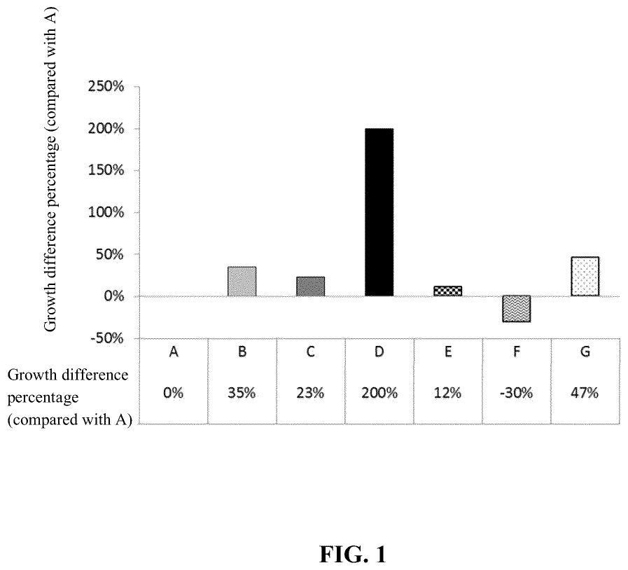

The Streptococcus thermophilus iHA318 strain of the present invention for growth bacterial count analysis in the medium containing the aforementioned different nutrient sources is shown in FIG. 1. It can be seen that compared with the medium A without adding the nutrient source, the medium D adding urea as the nutrient source can greatly increase the number of growth bacteria of the Streptococcus thermophilus iHA318 strain of the present invention by 200%. The rest of the medium can only increase the number of bacteria growth by 10-50%, and the medium F added with yeast extract would reduce the growth number of the Streptococcus thermophilus iHA318 strain of the present invention. This result shows that, compared with macromolecular nitrogen sources such as skimmed milk powder, tryptic soy, meat extract, yeast extract, casein, etc., the use of small molecular nitrogen sources such as urea is more suitable and optimum nutrient source for culturing the Streptococcus thermophilus iHA318 strain of the present invention. Small molecular nitrogen sources such as molecular nitrogen, ammonium sulfate and amino acids should be helpful for the growth of Streptococcus thermophilus iHA318 strain.

Next, in order to further understand the urea concentration most suitable for culturing the Streptococcus thermophilus iHA318 strain of the present invention, the original bacteria of the Streptococcus thermophilus iHA318 strain of the present invention in the frozen tube were thawed. 1 mL of bacterial solution was taken and 9 mL of sterile water was added. After standing for 1 hour, 10 μL of the diluted solution was taken and added to 10 mL of the medium D-1 to D-5 in Table 3, which contains 0%, 0.10%, 0.50%, 1.00%, or 5% urea, respectively. After quantifying with sterile water to a total volume of 1 L, incubation was performed at 37° C. for 24 hours, and the bacterial liquids of different media were mixed evenly and 10 mL of the mixture was taken. After adding 90 mL of sterile water to serially dilute to the appropriate concentration, 1 mL of the diluent was taken and the pour culture method was used to culture on MRS agar for 48 hours based on 37° C. anaerobic culture for bacterial count analysis.

TABLE 3

| Medium number | D-1 | | D-2 | | D-3 | | D-4 | | D-5 | |
|---|---|---|---|---|---|---|---|---|---|---|
| Urea concentration | 0% | | 0.10% | | 0.50% | | 1.00% | | 5% | |
| Proteose Peptone No3 | 10 | g | 10 | g | 10 | g | 10 | g | 10 | g |
| Beef Extract | 10 | g | 10 | g | 10 | g | 10 | g | 10 | g |
| Yeast Extract | 5 | g | 5 | g | 5 | g | 5 | g | 5 | g |
| Magnesium Sulfate | 0.1 | g | 0.1 | g | 0.1 | g | 0.1 | g | 0.1 | g |
| Dextrose (Glucose) | 20 | g | 20 | g | 20 | g | 20 | g | 20 | g |
| Polysorbate 80 | 1 | g | 1 | g | 1 | g | 1 | g | 1 | g |
| Ammonium Citrate | 2 | g | 2 | g | 2 | g | 2 | g | 2 | g |
| Sodium Acetate | 5 | g | 5 | g | 5 | g | 5 | g | 5 | g |
| Manganese Sulfate | 0.05 | g | 0.05 | g | 0.05 | g | 0.05 | g | 0.05 | g |
| Dipotassium Phosphate | 2 | g | 2 | g | 2 | g | 2 | g | 2 | g |
| urea | 0 | g | 1 | g | 5 | g | 10 | g | 50 | g |

| | |
|---|---|
| H₂O | Quantify to 1 L |

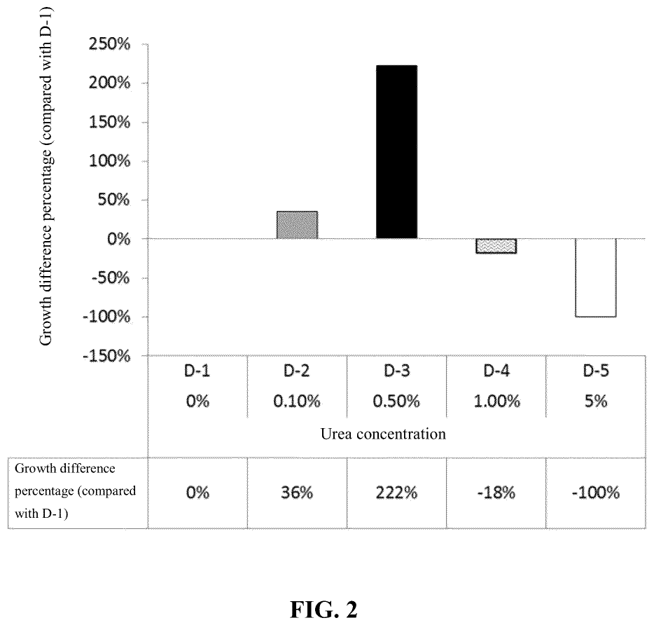

The Streptococcus thermophilus iHA318 strain of the present invention in the medium containing different concentrations of urea for the growth bacteria count analysis is shown in FIG. 2. It can be seen that compared with the culture medium D-1 without adding urea nutrient source, the culture medium D-3 adding urea as the nutrient source can greatly increase the number of growth bacteria of the Streptococcus thermophilus iHA318 strain of the present invention by more than 200%. The rest of the medium cannot effectively promote the growth of bacteria. This result shows that 0.5% urea concentration is the most suitable nutrient source concentration for culturing the Streptococcus thermophilus iHA318 strain of the present invention.

### Example 2

Antioxidant Effect on Nerve Cells of Streptococcus thermophilus iHA318 Strain of Present Invention

In one embodiment of the present invention, in order to test the antioxidant activity of the Streptococcus thermophilus iHA318 strain of the present invention on nerve cells, an oxidant was used to stimulate the nerve cell PC12 cell line treated with the iHA318 strain and its metabolites. The viability of the PC12 cells was tested to analyze the ability of the Streptococcus thermophilus iHA318 strain of the present invention to enhance the antioxidant activity of cells. In the embodiment of the present invention, hydrogen peroxide (H₂O₂) is used as the oxidant.

First, the cryovial of the Streptococcus thermophilus iHA318 strain of the present invention was taken out and then thawed, and the Streptococcus thermophilus iHA318 strain was inoculated into the MRS medium with a concentration of 1%. Incubation was performed at 37° C. for 16-24 hours in an anaerobic environment to activate the iHA318 strain. At the same time, PC12 cells were seeded at 1.5×10⁴/well in a 96-well culture dish, and each well contains 100 μL of culture medium. After culturing for 24 hours in 5% CO₂ at 37° C., the cell culture medium was removed for subsequent experiments.

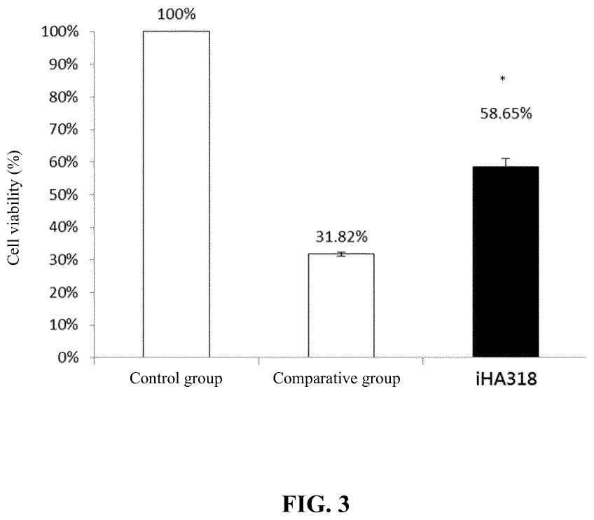

Next, 100 μL of the activated bacterial solution was taken out and placed in 10 mL of MRS medium, and cultivated in an anaerobic environment at 37° C. for 16-24 hours. The bacterial solution was diluted 20-50 times for later use, such as 20-25, 30-35, 35-40, 40-45, or 45-50 times dilution. The bacteria in the bacterial solution can be further removed or not removed, preferably by centrifugation or filtration to remove the bacteria, only the bacterial culture solution is retained, and three groups of PC12 cells are treated as follows. (1) Control group: treated with 100 μL of cell culture medium only, and not treated with hydrogen peroxide; (2) Comparative group: after adding 100 μL of cell culture medium and treating at 37° C. for 4 hours, the final concentration of 100 μM hydrogen peroxide (Sigma; prepared in cell culture medium) was treated for 24 hours; (3) Experimental group: after adding 100 μL of the bacterial culture solution of the aforementioned iHA318 strain and treating at 37° C. for 4 hours, the final concentration of 100 μM hydrogen peroxide (Sigma; prepared in cell culture medium) was treated for 24 hours. After removing the cell culture medium of each group, 100 μL of MTT reagent (3-(4,5-Dimethylthiazol-2-yl)-2,5-diphenyl tetrazolium bromide; Gibco) was added. After 2 hours at 37° C., it was removed, and 100 μL of dimethyl sulfoxide (DMSO) was added to react for 10 minutes. The absorbance of each group at 570 nm was detected to quantify the cell viability of PC12 cells. The result is shown in FIG. 3, in which the cell viability of the control group is 100%.

As can be seen from FIG. 3, compared with the control group without the reaction of hydrogen oxide, after the reaction of hydrogen oxide, the PC12 cells of the comparative group have only a cell viability of 31.82%. However, after treatment with the Streptococcus thermophilus iHA318 strain of the present invention, the PC12 cells of the experimental group treated with hydrogen oxide have a cell viability of 58.65%, which is significantly increased by 26.83% compared with the comparative group. This result shows that the Streptococcus thermophilus iHA318 strain of the present invention can effectively improve the cell viability of nerve cells in oxidative damage, so it has excellent antioxidant activity on nerve cells.

### Example 3

Antioxidant and Anti-Inflammatory Effects of Streptococcus thermophilus iHA318 Strain of Present Invention on Immune Cells

In one embodiment of the present invention, in order to test the antioxidant and anti-inflammatory activities of the Streptococcus thermophilus iHA318 strain of the present invention on immune cells, immune cells Raw264.7 cell line treated with iHA318 strain and its metabolites were stimulated with oxidants and pro-inflammatory agents, respectively. The cell viability of the Raw264.7 cells and the secretion amount of the inflammatory mediators secreted by the Raw264.7 cells were respectively tested to analyze the ability of the Streptococcus thermophilus iHA318 strain of the present invention to enhance the antioxidant and anti-inflammatory activities of the cells. In the example of the present invention, AAPH (2,2′-Azobis(2-amidinopropane) dihydrochloride) is used as the oxidant. Lipopolysaccharide (LPS) is used as a pro-inflammatory agent. The secretion of inflammatory mediator nitric oxide (NO) is detected using the Griess reagent.

First, the cryovial of the Streptococcus thermophilus iHA318 strain of the present invention was taken out and then thawed, and the Streptococcus thermophilus iHA318 strain was inoculated into the MRS medium with a concentration of 1%. Incubation was performed at 37° C. for 16-24 hours in an anaerobic environment to activate the iHA318 strain. Next, 100 μL of the activated bacterial solution was taken out and placed in 10 mL of MRS medium, and cultivated in an anaerobic environment at 37° C. for 16-24 hours. At the same time, Raw264.7 cells were seeded at 1×10⁴/well in a 96-well culture dish, and each well contains 100 μL of culture medium. After culturing for 24 hours in 5% CO₂ at 37° C., the cell culture medium was removed for subsequent experiments.

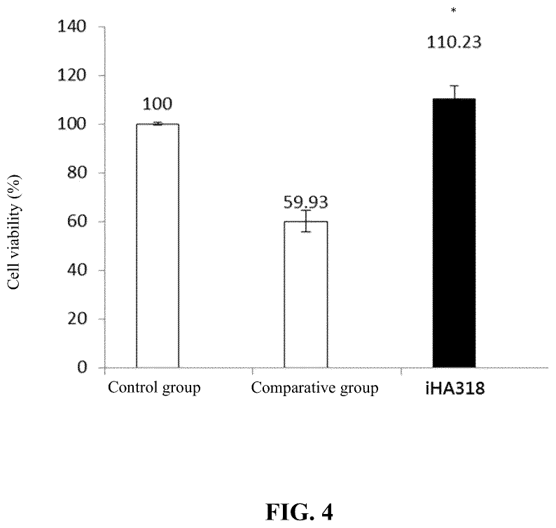

In the test of the antioxidant effect, the bacterial solution was diluted 80-100 times for later use, such as 80-85, 85-90, 90-95, or 95-100 times dilution. The bacteria in the bacterial solution can be further removed or not removed, preferably by centrifugation or filtration to remove the bacteria, only the bacterial culture solution is retained, and three groups of Raw264.7 cells are treated as follows. (1) Control group: treated with 100 μL of cell culture medium only, and not treated with AAPH; (2) Comparative group: after adding 100 μL of cell culture medium and treating at 37° C. for 4 hours, the final concentration of 60 mM AAPH (Sigma; prepared in cell culture medium) was treated for 24 hours; (3) Experimental group: after adding 100 μL of the bacterial culture solution of the aforementioned iHA318 strain and treating at 37° C. for 24 hours, the final concentration of 60 mM AAPH (Sigma; prepared in cell culture medium) was treated. After removing the cell culture medium of each group, 100 μL of MTT reagent (Gibco) was added. After 2 hours at 37° C., it was removed, and 100 μL of dimethyl sulfoxide (DMSO) was added to react for 10 minutes. The absorbance of each group at 570 nm was detected to quantify the cell viability of Raw264.7 cells. The result is shown in FIG. 4, in which the cell viability of the control group is 100%.

As can be seen from FIG. 4, compared with the control group without the reaction of AAPH, after the reaction of AAPH, the Raw264.7 cells of the comparative group have only a cell viability of 59.93%. However, after treatment with the Streptococcus thermophilus iHA318 strain of the present invention, the Raw264.7 cells of the experimental group treated with AAPH have a cell viability as the control group, which is significantly increased by 50.3% compared with the comparative group. This result shows that the Streptococcus thermophilus iHA318 strain of the present invention can effectively improve the cell viability of immune cells in oxidative damage, so it has excellent antioxidant activity on immune cells.

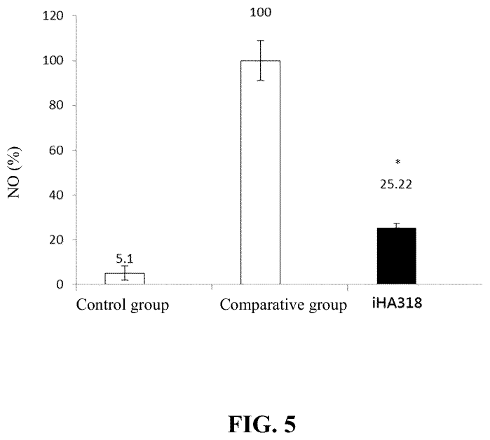

In the test of the anti-inflammatory effect, the bacterial solution was diluted 20-50 times for later use, such as 20-25, 25-30, 30-35, 35-40, 40-45, or 45-50 times dilution. The bacteria are removed by centrifugation or filtration, only the bacterial culture solution is retained, and three groups of Raw264.7 cells are treated as follows. (1) Control group: treated with 100 μL of cell culture medium only, and not treated with LPS; (2) Comparative group: after adding 100 μL of cell culture medium and treating at 37° C. for 24 hours, the final concentration of 1 μg/mL LPS (Sigma; prepared in cell culture medium) was treated for 48 hours; (3) Experimental group: after adding 100 μL of the bacterial culture solution of the aforementioned iHA318 strain and treating at 37° C. for 24 hours, the final concentration of 1 μg/mL LPS (Sigma; prepared in cell culture medium) was treated for 48 hours. 50 μL of cell supernatant from each group was collected, and 50 μL of Griess reagent (Sigma) was added to react for 15 minutes. If the cell supernatant contains nitric oxide, the solution would appear purple. Therefore, the absorbance at 550 nm of each group was detected to quantify the concentration of nitric oxide secreted by Raw264.7 cells to indicate the stimulated inflammatory response of the cells. The result is shown in FIG. 5, in which the nitric oxide concentration of the control group is 100%.

As can be seen from FIG. 5, if the nitric oxide secretion of the Raw264.7 cells of the comparative group after the reaction of LPS is defined as 100%, after treatment with the Streptococcus thermophilus iHA318 strain of the present invention, the Raw264.7 cells of the experimental group treated with LPS only have a nitric oxide secretion of 25.22%. Compared with the comparative group, the ratio can be significantly reduced by 74.74%. This result shows that the Streptococcus thermophilus iHA318 strain of the present invention can effectively reduce the inflammatory response induced by immune cells, so it has excellent anti-inflammatory activity on immune cells.

### Example 4

Effect of Streptococcus thermophilus iHA318 Strain of Present Invention on Alleviating Dry Eye Syndrome

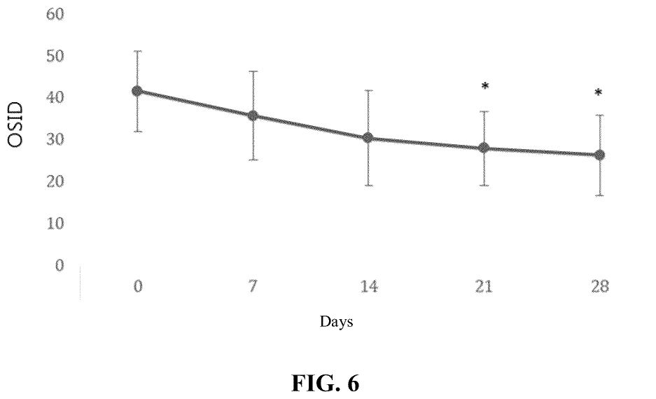

In one embodiment of the present invention, in order to test the effect of the Streptococcus thermophilus iHA318 strain of the present invention on alleviating dry eye syndrome in the subject, 5 subjects aged 25-40 with dry eye syndrome are recruited. 50 mg of Streptococcus thermophilus iHA318 strain of the present invention was taken daily (equivalent to daily intake of 1×10⁸-1×10¹¹ CFU) for a total of 28 days. The ocular surface disease index scale (OSDI, an international scale commonly used to evaluate dry eye syndrome) was filled in on the 0^{th}, 7^{th}, 14^{th}, 21^{st} and 28^{th} days of administration, to evaluate the effect of the Streptococcus thermophilus iHA318 strain of the present invention on preventing and/or treating dry eye syndrome in the subject. The result is shown in FIG. 6.

As can be seen from FIG. 6, as the days of taking the Streptococcus thermophilus iHA318 strain of the present invention increase, the symptoms of dryness and discomfort on the ocular surface of the subject would gradually ease. After 28 days of use, it can reduce the dryness and discomfort of the ocular surface by 36.4%. All participants were improved, with an average OSID reduction of 36.4%. This result shows that the Streptococcus thermophilus iHA318 strain of the present invention can effectively relieve the discomfort of the ocular surface of individuals, and can be effectively used for the prevention and/or treatment of dry eye syndrome.

### Example 5

Effect of Streptococcus thermophilus iHA318 Strain of Present Invention on Secreting Sialic Acid

In one embodiment of the present invention, in order to test the effect of the Streptococcus thermophilus iHA318 strain of the present invention to secrete sialic acid, the amount of sialic acid secreted by the iHA318 strain was analyzed by colorimetry. The differences in the ability to secrete sialic acid between Streptococcus thermophilus ST002 strain, which is also isolated from breast milk samples, and the Streptococcus thermophilus BCRC 14017 strain purchased from the Food Industry Research and Development Institute (FIRDI), are compared.

First, the cryovials of the Streptococcus thermophilus ST002 strain, the Streptococcus thermophilus BCRC 14017 strain, and the Streptococcus thermophilus iHA318 strain of the present invention were taken out and then thawed, and strains were inoculated into the MRS medium or M17 liquid medium with a concentration of 1%. Incubation was performed at 37° C. for 16-24 hours in an anaerobic environment to activate the three strains. 100 μL of the activated bacterial solution was taken out and placed in 10 mL of MRS medium or M17 liquid medium, and cultivated in an anaerobic environment at 37° C. for 16-24 hours.

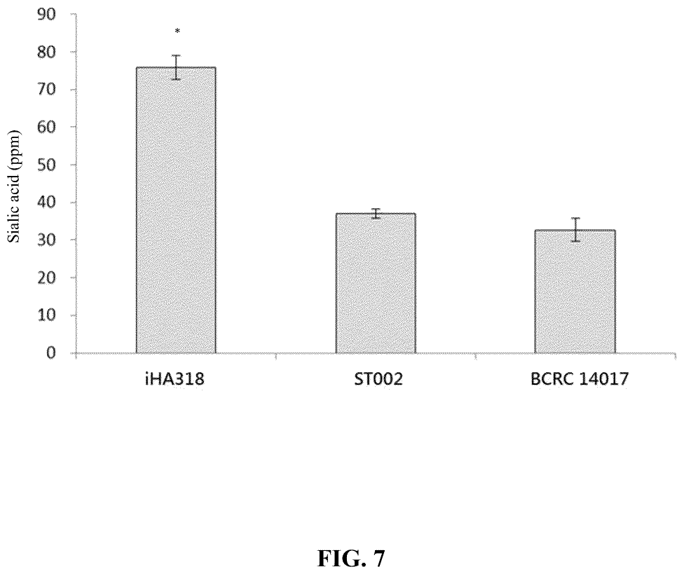

Equal amount of bacterial solution was respectively taken out and analyzed. The sialic acid can be purified and then quantified for its secretion, or the sialic acid content can be directly quantified after pretreatment of the bacterial solution. Bacterial solution can be directly used for the purification of sialic acid, or bacterial culture solution can be used (bacterial cells in bacterial solution were removed by centrifugation). The bacterial solution or bacterial culture solution was hydrolyzed and desalted, and then ion exchange resin was used to purify the pure sialic acid. In the embodiment of the present invention, 4 mL of bacterial solution was taken out respectively for the pretreatment of sialic acid analysis. 50 μL of 8N hydrogen chloride (HCl) was added, mixed evenly, and reacted at 80° C. for 2 hours. After cooling, 50 μL of sodium hydroxide (NaOH) was added and mixed evenly. 40 μL of the solution was taken out after proper dilution as the sample to be tested for the content analysis of sialic acid. 40 μL of N-Acetylneuraminic acid was also taken as the standard. 20 μL of 0.2M sodium periodate (NaIO₄) (dissolved in 9M phosphoric acid (H₃PO₄)) was added to the test sample and standard, and reaction was performed at room temperature for 20 minutes. 200 μL of a solution containing 10% sodium metaarsenite (NaAsO₂) and 0.2M Na₂IO₄ (containing 0.01% KI, dissolved in 0.1M sulfuric acid (H₂SO₄)) was added, and mixed well until the brown color disappears. 600 μL of 0.6% thiobarbituric acid (aqueous solution containing 7% sodium sulfate (Na₂SO₄)) was added, mixed well and reacted at 100° C. for 15 minutes. After cooling, 400 μL of cyclohexanone was added, followed by centrifugation at 12,000 g for 5 minutes. The supernatant was taken out and the absorbance was measured at 549 nm. A standard curve was plotted with the absorbance value of the standard, and the sialic acid concentration of the sample to be tested was calculated by the interpolation to obtain the sialic acid concentrations secreted by the Streptococcus thermophilus ST002 strain, the BCRC 14017 strain, and the iHA318 strain of the present invention. The result is shown in FIG. 7.

As can be seen from FIG. 7, the Streptococcus thermophilus ST002 strain and the BCRC 14017 strain can only secrete about 35 ppm of sialic acid. However, by culturing the strain in the same way, the Streptococcus thermophilus iHA318 strain of the present invention can produce about 75 ppm of sialic acid, which is more than 2 times that of the other two strains. This result shows that the Streptococcus thermophilus iHA318 strain of the present invention can secrete sialic acid more effectively, and can be applied to the production of sialic acid and the use of skin health and health care.

### Example 6

Effect of Streptococcus thermophilus iHA318 Strain of Present Invention on Secreting Hyaluronic Acid

In one embodiment of the present invention, in order to test the effect of the Streptococcus thermophilus iHA318 strain of the present invention to secrete hyaluronic acid, the amount of hyaluronic acid secreted by the iHA318 strain was analyzed by colorimetry. The differences in the ability to secrete hyaluronic acid between Streptococcus thermophilus ST002 strain and the Streptococcus thermophilus BCRC 14017 strain are compared.

First, the cryovials of the Streptococcus thermophilus ST002 strain, the Streptococcus thermophilus BCRC 14017 strain, and the Streptococcus thermophilus iHA318 strain of the present invention were taken out and then thawed. The three strains were activated as described in Example 4. 100 μL of the activated bacterial solution was taken out and placed in 10 mL of MRS medium or M17 liquid medium, and cultivated in an anaerobic environment at 37° C. for 16-24 hours.

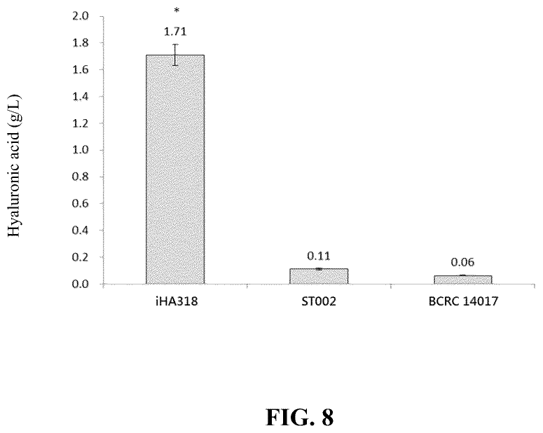

10 mL of bacterial solution was taken out for purification of hyaluronic acid. After adding 40 mL of 95% alcohol and mixing well, centrifugation was performed at 5000 rpm for 10 minutes and the supernatant was removed. 40 mL of 95% alcohol was added again and mixed well. Centrifugation was performed at 5000 rpm for 10 minutes, the supernatant was removed, followed by quantifying to 10 mL with water. 100 μL of the solution was taken out after proper dilution as the sample to be tested for the content analysis of hyaluronic acid. 100 μL of glucuronic acid was also taken as the standard. The sample was slowly added to 600 μL of borax sulfuric acid solution in an ice bath, mixed evenly, and then placed in a 100° C. water bath to react for 10 minutes. After cooling to room temperature, the reactant was put in an ice bath, and then 20 μL of carbazole solution was added. After mixing well, the reactant was placed in a 100° C. water bath for 15 minutes. After cooling to room temperature and mixing well, 100 μL of the reaction solution was taken into a 96-well plate. The absorbance was measured at 525 nm. A standard curve was plotted with the absorbance value of the standard, and the hyaluronic acid concentration of the sample to be tested was calculated by the interpolation, followed by multiplying the dilution multiple and the conversion factor of 2.07 to obtain the hyaluronic acid concentrations secreted by the Streptococcus thermophilus ST002 strain, the BCRC 14017 strain, and the iHA318 strain of the present invention. The result is shown in FIG. 8.

As can be seen from FIG. 8, the Streptococcus thermophilus ST002 strain and the BCRC 14017 strain can only secrete 0.11 g/L and 0.06 g/L hyaluronic acid. However, by culturing the strain in the same way, the Streptococcus thermophilus iHA318 strain of the present invention can produce 1.71 g/L hyaluronic acid, which is more than 15 times that of the other two strains. This result shows that the Streptococcus thermophilus iHA318 strain of the present invention can secrete hyaluronic acid more effectively, and can be applied to the production of hyaluronic acid and the use of skin health and health care.

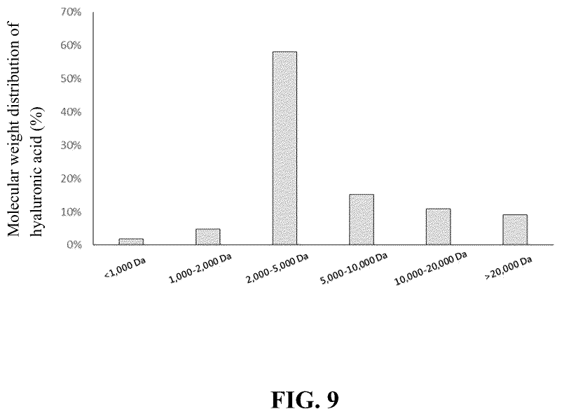

To further understand the molecular weight of the hyaluronic acid secreted by the Streptococcus thermophilus iHA318 strain of the present invention, after the aforementioned iHA318 strain was cultured and purified by hyaluronic acid, it was adjusted to an appropriate concentration and the molecular weight was measured by conventional means. The result is shown in FIG. 9, in which it can be seen that the molecular weights of hyaluronic acid secreted by the Streptococcus thermophilus iHA318 strain of the present invention are: <1,000 Da: 1.90%; 1,000-2,000 Da: 4.70%; 2,000-5,000 Da: 58.10%; 5,000-10,000 Da: 15.30%; 10,000-20,000 Da: 10.80%; >20,000 Da: 9.20%, and have a certain effect in this interval. This result shows that the Streptococcus thermophilus iHA318 strain of the present invention can secrete hyaluronic acid with a wide molecular weight range, and can be applied to beauty and health care of different parts such as skin, eyes and joints.

In summary, the present invention provides a novel Streptococcus thermophilus iHA318 strain, and the optimum culture formulation of the Streptococcus thermophilus iHA318 strain is measured, to enhance the activity of the iHA318 strain and greatly increase the number of bacterial growth. The Streptococcus thermophilus iHA318 strain and/or its metabolites of the present invention can effectively improve the cell viability of nerve cells and immune cells in oxidative damage, and has excellent antioxidant activity for nerve cells and immune cells. At the same time, it can effectively reduce the inflammatory response triggered by immune cells, so it has excellent anti-inflammatory activity on immune cells. Compared to the Streptococcus thermophilus ST002 strain also isolated from breast milk and the conventional Streptococcus thermophilus BCRC 14017 strain, the novel Streptococcus thermophilus iHA318 strain of the present invention can more effectively secrete sialic acid and hyaluronic acid. Therefore, it can be used in the production of sialic acid and hyaluronic acid, and in moisturizing, immune regulation, dry eye syndrome, antioxidant and anti-inflammatory purposes.

### Example 7

Animal Experiment of Streptococcus thermophilus iHA318 Strain of Present Invention for Relieving Dry Eye Syndrome

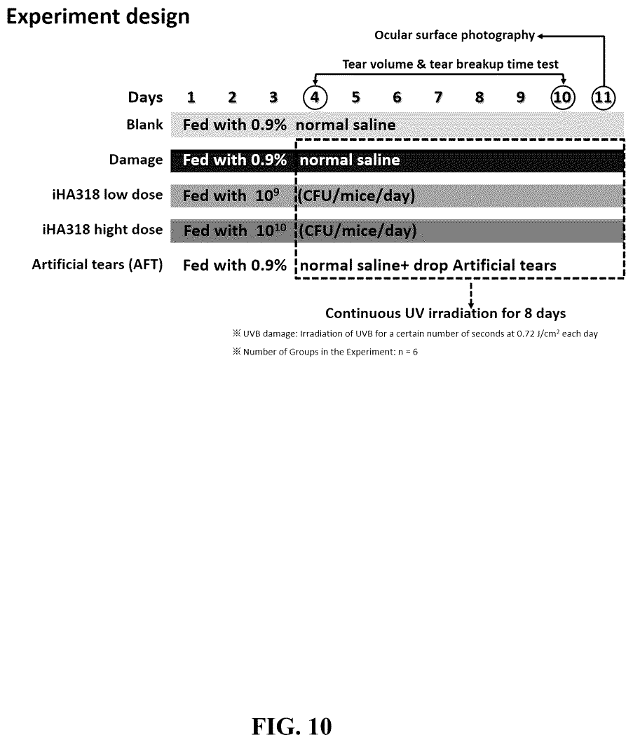

The research materials and instruments of this example are as follows: (1) thirty 6-week-aged ICR female mice (BioLASCO Taiwan Co., Ltd.); (2) sample: iHA318 bacterial powder; (3) 2.5% Avertin anesthetic; (4) Lissamine™ Green B corneal stain (Sigma 199583); (5) normal saline; (6) artificial tears; (7) corneal smoothness tester. The experimental animals were ICR female mice purchased from BioLASCO Taiwan Co., Ltd., aged 7-10 weeks and weighing 25-33 g. The animals were raised in the Laboratory Animal Center of Chung Shan Medical University, providing normal feed and drinking water. The living environment is 12 hours of light and 12 hours of dark in cycle, the temperature is controlled at 20±2° C., and the humidity is controlled at 65±5%. The mice were randomly divided into 5 groups with 6 mice in each group, which were the control group (fed with 0.9% normal saline solution), the damage group (treated with 0.9% normal saline solution and UVB), the iHA318 low dose group (treated with UVB and fed daily tube feeding with 1.0*10⁹ iHA318 strain), the iHA318 high dose group (treated with UVB and fed daily tube feeding with 1.0*10¹⁰ iHA318 strain) and artificial tears group (treated with UVB and administered artificial tears daily and fed 0.9% normal saline solution). The UV lamp was purchased from Vilber lourmat, Germany, the lamp model was VL-6MC, the wavelength was set in the range of 280 nm to 320 nm, and the main peak was at 312 nm. The experimental process was 11 days in total. The test samples were fed daily from the first three days of UV irradiation, and continued to be given until the end of the test. Damage group, iHA318 low dose group, iHA318 high dose group and artificial tears group were irradiated with UVB from the 4th day to the 11th day. Mice were anesthetized with 2.5% Avertin daily and placed in a dark box. UVB was irradiated with an intensity of 0.72 J/cm² for 90 seconds with the eyeball facing upward, which damaged the ocular surface of mice and induced dry eye syndrome. Tear volume (TV) and tear film break up time (TBUT) tests were performed on the 3^{rd} and 10th days, respectively. Ocular Surface Photography was performed on day 11. The above experimental procedure is shown in FIG. 10.

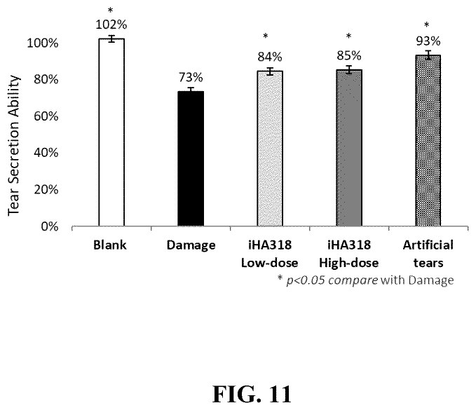

The analysis of dry eye syndrome is as follows: (1) Tear test: in this experiment, the tear secretion of mice was tested on the third and tenth day (the seventh day of continuous UV irradiation). Litmus paper was used as a test tool, and the litmus paper was cut with a width of 7 mm into thin strips of 1 mm width each. After the mice were anesthetized, litmus paper was placed in the eye socket next to the ear to draw tears. After 20 seconds, the litmus paper was taken out to measure the length and to measure the tear secretion amount of mice. When mice with dry eye syndrome were damaged, their tear volume was significantly reduced to assess whether the mice had altered tear secretion. (2) Tear film break up time (TBUT): in this experiment, the tear film break up time of mice was tested on the third and tenth day (the seventh day of continuous UV irradiation). The tear film break up time refers to the maintenance time of the tear fluid on the cornea from completely covering the surface to the beginning of a hole after blinking. This test can initially detect the quality of tears. If the tear quality is good, the tear film can be maintained for a certain period of time. If the tear quality is not good, the time would be reduced. (3) Corneal appearance analysis: corneal smoothness test, corneal opacity test, corneal topographic test and corneal staining test were performed. Higher rating scores indicate more damage to the cornea. (a) Corneal smoothness: the surface of the eyeball is illuminated with a ring light source, and graded according to the completeness of the ring image of the corneal reflected light source, divided into grades 0 to 5; grade 0 images are complete and undistorted rings, grades 1-3 are ¼, ½, and ¾ parts of the ring are distorted in sequence, grade 4 is the distortion of the entire ring, and the worst is grade 5 is so distorted that the ring line cannot be recognized. (b) Corneal opacity: irradiate the eyeball with a light source to observe the clarity of the cornea, graded from 0 to 4 according to the degree of opacity. Grade 0 is normal corneal transparency. Grades 1-3 are mild, moderate, and moderate (unclear iris) opacity, respectively. Grade 4 is severe opacity, and obvious white cloudiness can be observed. (c) Corneal topography analysis: corneal topography is a projection of the ocular surface in a circular shape, which can observe a wide range of corneal smoothness. The evaluation method is to divide the ocular surface into 4 areas with a cross, and each area has 5 arcs in 5 circles. Count 1 point each time a circular arc line is distorted or cannot be interpreted, total 20 points for the whole eye. Higher scores indicate higher corneal unsmooth. According to the degree, it is divided into 0-grades, and the score is 0 point as grade 0. 1-4 is divided into grade 1, 5-9 is divided into grade 2, 10-14 is divided into grade 3, 15-19 is divided into grade 4, and the most severe 20 is divided into grade 5. (d) Corneal staining: since the damaged cornea would be stained with dyes, the degree of corneal damage can be assessed by the staining area. The analysis result is rated as 0-5 according to the size of the area. Unstained is grade 0, less than 25% is grade 1, 25-50% is grade 2, 50-75% is grade 3, 75%-99% is grade 4, and all corneas are stained as grade 5. The result is shown in FIGS. 11-16.

FIG. 11 shows that the Streptococcus thermophilus iHA318 strain of the present invention significantly restores tear secretion capacity, in which *p<0.05 compared with Damage group. UV-damaged mice significantly reduce tear secretion. According to the experimental result, the iHA318 strain can significantly increase the amount of tears and restore the function of tear secretion.

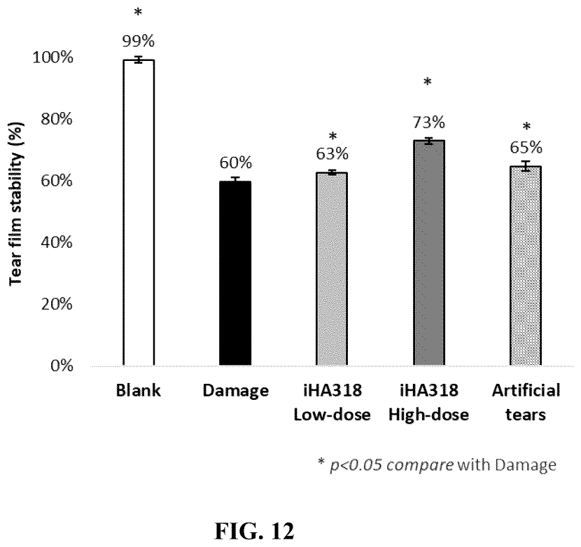

FIG. 12 shows that the Streptococcus thermophilus iHA318 strain of the present invention gradually restores tear film function. By analyzing tear film break up time, it is estimated that after blinking, the time between the complete coverage of the surface of the cornea and the appearance of a hole in the cornea. In UV-damaged mice, tear film break up time is significantly reduced. According to the experimental result, the iHA318 strain gradually repairs the tear film function and improves the tear film break up time.

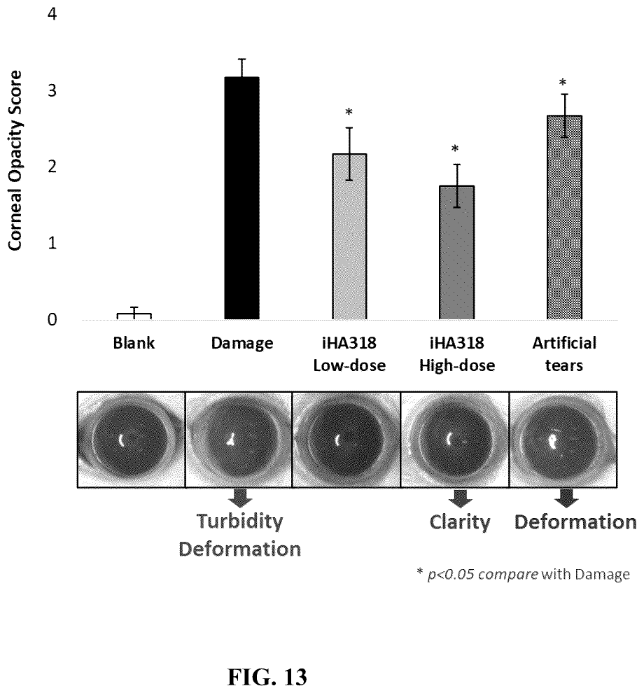

FIG. 13 shows that the Streptococcus thermophilus iHA318 strain of the present invention repairs and maintains eye transmittance. The eyeball was irradiated with a light source to observe the clarity of the cornea and whether the ¼ circle aperture can be maintained. In damaged mice, the cornea has poor light transmission, which scatters the light and turns white and cloudy. After using the iHA318 strain, light refraction and eye clarity are maintained, and the effect is better than artificial tears.

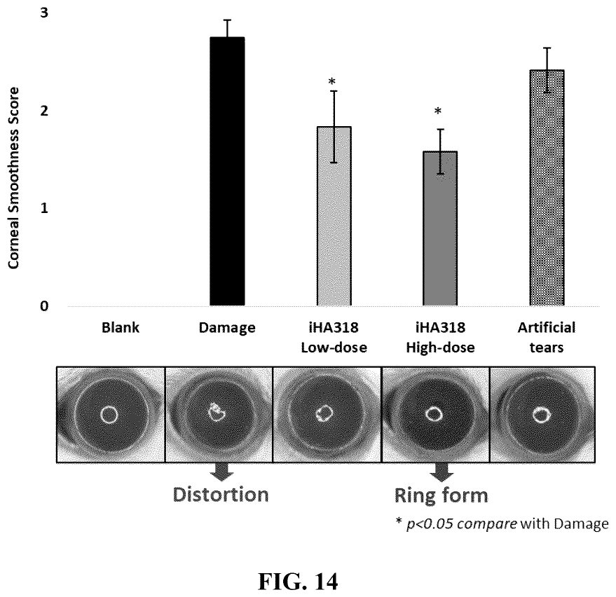

FIG. 14 shows that the Streptococcus thermophilus iHA318 strain of the present invention repairs pupil surface smoothness and maintains clear vision. The pupil is illuminated with a ring light source. The closer the aperture is to the circle, the better the smoothness. Severe distortion of the ring is occurred in damaged mice, which also causes blurred vision. After using the iHA318 strain, it can repair the distortion caused by external damage, maintain a clear vision, and the effect is better than artificial tears.

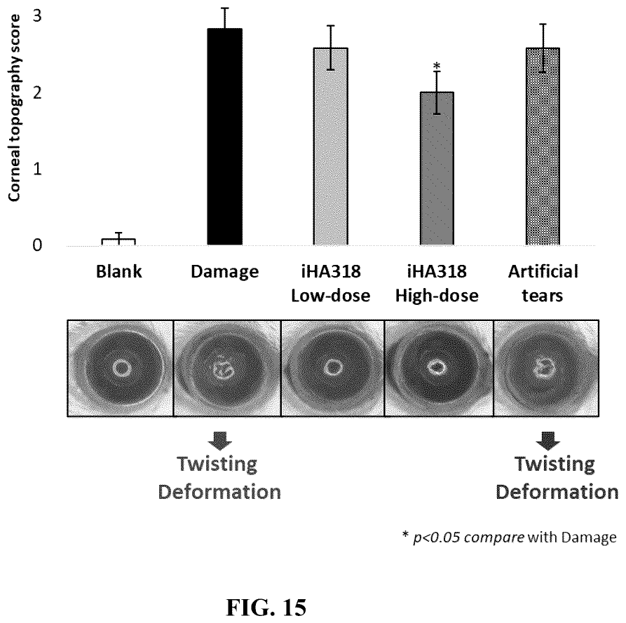

FIG. 15 shows that the Streptococcus thermophilus iHA318 strain of the present invention repairs and maintains the smoothness of the entire ocular surface, and protects the health of the ocular surface in an all-round way. The corneal topography image is a circular projection of the ocular surface to observe the smoothness of the cornea in a wide range. The more complete the concentric circles, the smoother the cornea. In the damaged mice, the concentric circles were severely distorted, showing extensive damage to the ocular surface. The result shows that the iHA318 strain can repair and maintain the smoothness of the entire ocular surface, protect the health of the ocular surface in an all-round way, and the effect is better than artificial tears.

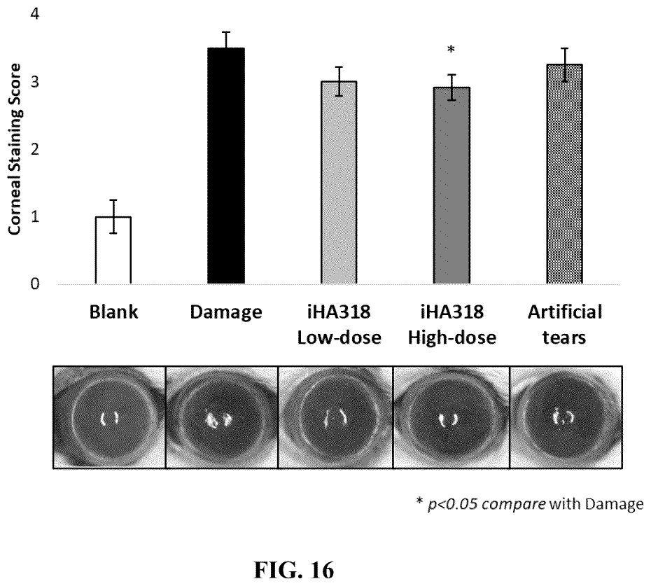

FIG. 16 shows that the Streptococcus thermophilus iHA318 strain of the present invention repairs ocular surface damage. In damaged mice, the damaged cornea would be adsorbed and colored by the dye to observe the degree of ocular surface damage. After using the iHA318 strain, the adsorption of dyes on the ocular surface is reduced, showing that it helps to repair ocular surface damage, and the effect is better than artificial tears.

Based on the above experimental results, although artificial tears can maintain the ability of tear secretion and restore the function of tear film, the repair ability of ocular surface damage is poor, and it can only temporarily relieve dry eyes. After using the iHA318 strain, it can increase the secretion of tears and maintain the function of tear film, and can also slow down the damage to the ocular surface caused by external stimuli.

Although the present invention has been described with reference to the preferred embodiments, it will be apparent to those skilled in the art that a variety of modifications and changes in form and detail may be made without departing from the scope of the present invention defined by the appended claims.

What is claimed is:

1. A Streptococcus thermophilus iHA318 strain, deposited in Deutsche Sammlung von Mikroorganismen und Zellkulturen (DSMZ) under an accession number DSM 33978, wherein the Streptococcus thermophilus iHA318 strain produces sialic acid.

2. A probiotic composition, comprising a Streptococcus thermophilus iHA318 strain and/or its metabolites, wherein the Streptococcus thermophilus iHA318 strain is deposited in Deutsche Sammlung von Mikroorganismen und Zellkulturen (DSMZ) under an accession number DSM 33978.

3. The probiotic composition according to claim 2, wherein the Streptococcus thermophilus iHA318 strain is cultivated using a medium, and the medium comprises a nutrient source which is a small molecular nitrogen source.

4. The probiotic composition according to claim 3, wherein the small molecular nitrogen source is urea.

5. The probiotic composition according to claim 2, wherein the Streptococcus thermophilus iHA318 strain is viable, inactive or its metabolites.

6. A method for producing sialic acid and/or hyaluronic acid by using the probiotic composition according to claim 2.

7. The method according to claim 6, wherein the probiotic composition is a medicament, a nutritional supplement, a health food, a food product, a skin health product, an external product, or a combination thereof.

8. A method for anti-oxidation, comprising administering to a subject in need thereof a pharmaceutical composition comprising an effective amount of the probiotic composition according to claim 2.

9. The method according to claim 8, wherein the probiotic composition enhances antioxidant ability of a nerve cell and/or an immune cell.

10. The method according to claim 8, wherein the pharmaceutical composition is a medicament, a nutritional supplement, a health food, a food product, a skin health product, an external product, or a combination thereof.

11. A method for anti-inflammation, comprising administering to a subject in need thereof a pharmaceutical composition comprising an effective amount of the probiotic composition according to claim 2.

12. The method according to claim 11, wherein the probiotic composition reduces an inflammatory response of an immune cell.

13. The method according to claim 11, wherein the pharmaceutical composition is a medicament, a nutritional supplement, a health food, a food product, a skin health product, an external product, or a combination thereof.

14. A method for preventing and/or treating dry eye syndrome, comprising administering to a subject in need thereof a pharmaceutical composition comprising an effective amount of the probiotic composition according to claim 2.

15. The method according to claim 14, wherein the effective amount is at least 1×10⁸-1×10¹¹ CFU of the Streptococcus thermophilus iHA318 strain.

16. The method according to claim 14, wherein the pharmaceutical composition is a medicament, a nutritional supplement, a health food, a food product, a skin health product, an external product, or a combination thereof.


SEQUENCE LISTING (7 sequences)

SEQ ID NO: 1 - DNA, 20 bp, synthetic construct
  misc_feature 1..20: note = synthetic primer
  source 1..20: mol_type = other DNA
agagtttgatcmtggctcag

SEQ ID NO: 2 - DNA, 17 bp, synthetic construct
  misc_feature 1..17: note = synthetic primer
  source 1..17: mol_type = other DNA
aaggaggtgwtccarcc

SEQ ID NO: 3 - DNA, 23 bp, synthetic construct
  misc_feature 1..23: note = synthetic primer
  source 1..23: mol_type = other DNA
tggagagtttgatcctggctcag

SEQ ID NO: 4 - DNA, 20 bp, synthetic construct
  misc_feature 1..20: note = synthetic primer
  source 1..20: mol_type = other DNA
ggactaccagggtatctaat

SEQ ID NO: 5 - DNA, 20 bp, synthetic construct
  misc_feature 1..20: note = synthetic primer
  source 1..20: mol_type = other DNA
agagtttgatcytggytyag

SEQ ID NO: 6 - DNA, 20 bp, synthetic construct
  misc_feature 1..20: note = synthetic primer
  source 1..20: mol_type = other DNA
ctttacgcccartrawtccg

SEQ ID NO: 7 - DNA, 755 bp, Streptococcus thermophilus
  source 1..755: mol_type = genomic DNA
gcgtgctatacatgcaagtagaacgctgaagagaggagcttgctcttcttggatgagttgcgaacgggtgagtaacgcgtaggtaacctgccttgtagcgggggataactattggaaacgatagctaataccgcataacaatggatgacacatgtcatttatttgaaaggggcaattgctccactacaagatggacctgcgttgtattagctagtaggtgaggtaatggctcacctaggcgacgatacatagccgacctgagagggtgatcggccacactgggactgagacacggcccagactcctacgggaggcagcagtagggaatcttcggcaatgggggcaaccctgaccgagcaacgccgcgtgagtgaagaaggttttcggatcgtaaagctctgttgtaagtcaagaacgggtgtgagagtggaaagttcacactgtgacggtagcttaccagaaagggacggctaactacgtgccagcagccgcggtaatacgtaggtcccgagcgttgtccggatttattgggcgtaaagcgagcgcaggcggtttgataagtctgaagttaaaggctgtggctcaaccatagttcgctttggaaactgtcaaacttgagtgcagaaggggagagtggaattccatgtgtagcggtgaaatgcgtagatatatggaggaacaccggtggcgaaagcggctctctggtctgtaactgacgctgaggctcgaaagcgtggggagcgaacaggattagt